(19) United States
(12) Patent Application Publication
(10) Pub. No.: US 2006/0166872 A1
(43) Pub. Date: Jul. 27, 2006

(54) Fp receptor antagonists or pgf2 alpha antagonists for treating menorrhagia

(76) Inventors: Henry Nicolas Jabbour, Edinburgh (GB); Hilary Octavia Dawn Critchley, Edinburgh (GB); Stuart Angus Milne, Edinburgh (GB)

Correspondence Address: Edwin V Merkel;Nixon Peabody, Clinton Square, P O Box 31051, Rochester, NY, 14603-1051, US

(21) Appl. No.: 10/511,484

(22) PCT Filed: Apr. 10, 2003
(86) PCT No.: PCT/GB03/01536

§ 371 Date: Oct. 21, 2005

(30) Foreign Application Priority Data
Apr. 17, 2002 (GB) 0208785.6
Apr. 17, 2002 (GB) 0208783.1

(51) Int. Cl. A61K 38/10; A61K 38/08; A61K 31/557

(52) U.S. Cl. 514/12; 514/13; 514/14; 514/15; 514/16; 514/573

(57) ABSTRACT

A method of treating or preventing menorrhagia in an female individual the method comprising administering to the individual at least one agent that prevents PGP_{2α} having its effect on the FP receptor. Optionally, an inhibitor of PGES and/or an antagonist of EP2 or EP4 is also administered.

The present invention relates to methods of treatment, and in particular methods of treating menorrhagia.

Menorrhagia is over-abundance of the menstrual discharge.

Menorrhagia affects many women, particularly in the Western world, and represent a significant health problem. At least one in 20 women in the UK aged between 34 and 49 years will consult their general practitioners because of menstrual problems. These women account for more than one in ten of all gynaecological referrals and cost the NHS in excess of £7 million per year for medical prescriptions alone. Perceived abnormal vaginal bleeding is said to account for 70% of the at least 70,000 hysterectomies done each year.

At present, the treatments used for menorrhagia include tranexamic acid or mefenamic acid. In severe cases the treatment is hysterectomy (vaginal or abdominal) but this is a major operation with serious morbidity and some risk of death. A review of treatments for menorrhagia is Stirrat (1999) The Lancet 353, 2175-2176. The development of further and alternative therapies is desirable.

The FP prostaglandin receptor has been studied in a variety of tissues including the bovine corpus luteum (Sharif et al 1998, J. Pharmacol. Exp. Ther. 286: 1094-1102); human uterus (Senior et at 1992, Br. J. Pharmacol. 108: 501-506); rabbit jugular vein (Chen et al 1995, Br. J. Pharmacol 116: 3035-3041); various human ocular tissues (Davis & Sharif 1999, J. Ocular Pharmacol. Ther. 15: 323-336); and in mouse Swiss 3T3 fibroblasts (Griffin et at 1997, J. Pharmacol. Exp. Ther. 281: 845-854); and in rat vascular smooth muscle cells (A7r5) Griffin et at 1998, J. Pharmacol. Exp. Ther. 286: 411-418).

Potent, selective synthetic agonists at some prostaglandin receptors have been characterised in both in vitro and in vivo models (Coleman et at 1994, Pharmacol. Rev. 46: 205-229). For instance fluprostenol or its enantiomer (eg AL-5848) (Sharif et al. 1999, J. Pharm. Pharmacol., 51: 685-694) and cloprostenol (Coleman et al 1994; Sharif et al 1998) are potent and selective FP receptor agonists. Since most natural prostaglandins show rather limited selectivity for their preferred receptor among this receptor family, the few reported selective prostaglandin receptor agonists have been very valuable tools for discriminating discrete functional responses coupled to their respective receptors. However, conclusive identification of the particular receptors mediating prostaglandin-stimulated functional responses requires potent and selective antagonists (Kenakin 1996, Pharmacol. Rev. 48: 413:463).

The recent identification and commercial development of selective FP receptor agonists as potent and highly efficacious drugs for the treatment of elevated intraocular pressure (Bito 1997, Surv. Ophthalmol. 41 (Suppl. 22): S1-S14; Hellberg et al 1998, Invest. Ophthalmol. Vis. Sci. 39 (Suppl.): 1961) has considerably advanced our knowledge of FP receptor-coupled pharmacological actions. However, the function of the FP receptor is not fully understood, due in part to significant species differences in the tissue distribution of this receptor (Ocklind et al 1996, Invest. Ophthalmol. Vis. Sci. 37: 716-726; Davis & Sharif 1999; Sharif et al 1999).

Griffin et al (J. Pharmacol. Exp. Ther. 1999, 290: 1278-1284) reported the discovery of a selective FP receptor antagonist (AL-8810) of micromolar potency. Sharif et al (J. Pharm. Pharmacol. 2000, 52: 1529-1539) describe another analogue of PGF_{2α} (AL-3138; Ro-22-6641; 11-deoxy-16-fluoro PGF_{2α}) which is a partial agonist of low efficacy and which also functions as an FP receptor antagonist. AL-3138 being a relatively selective agent may be a valuable FP receptor antagonist tool for investigating the specific function of the FP receptor in various biological systems.

Cyclooxygenase (COX) enzymes, also called prostaglandin endoperoxide synthase (PGHS), catalyse the rate limiting step in the conversion of arachidonic acid to prostaglandin H₂ (PGH₂). In turn PGH₂ serves as a substrate for specific prostaglandin synthase enzymes that synthesise the natural prostaglandins. These are named according to the prostaglandin they produce such that prostaglandin D₂ is synthesised by prostaglandin-D-synthase, prostaglandin E₂ (PGE₂) by prostaglandin-E-synthase (PGES) and prostaglandin F_{2α} (PGF_{2α}) by prostaglandin-F-synthase (PGFS). To date, there are two identified isoforms of the COX enzyme, COX-1 and COX-2 (DeWitt, 1991). COX-1 is constitutively expressed in many tissues and cell types and generates prostaglandins for normal physiological function (Herschman, 1996). By contrast, the expression of COX-2 is rapidly induced following stimulation of quiescent cells by growth factors, oncogenes, carcinogens and tumour-promoting phorbol esters (Herschman, 1996; Subbaramaiah et al., 1996).

We have shown that expression of the PGF_{2α} receptor in the uterus across the menstrual cycle demonstrates higher levels of receptor expression during the proliferative phase of the endometrium compared with other stages. Expression in uterine carcinoma tissue is significantly elevated compared with normal uterine tissue. Using an endometrial epithelial cell line, we have demonstrated that PGF_{2α} induces proliferation of epithelial cells. This proliferation can be inhibited by using specific inhibitors of the PLC signalling pathway. We have also shown that the level of FP receptor present in endometrial tissue in women with menorrhagia is greatly increased compared to normal tissue controls.

These observations demonstrate the possibility of antagonising the PGF_{2α} (FP) receptor to combat menorrhagia.

Antagonists of the FP receptor have been suggested for treating or preventing premature delivery of a foetus and dysmenorrhoea, acting by the mechanism of relaxation of smooth muscle (WO 99/32640 and WO 00/17348). They have not, however, been previously suggested to be useful in combating menorrhagia.

A first aspect of the invention provides a method of treating or preventing menorrhagia in a female individual, the method comprising administering to the individual at least one agent that prevents PGF_{2α} having its effect on the FP receptor.

It is believed that in some cases menorrhagia may be associated with overproliferation of the uterine epithelium.

The female individual may be any individual or patient who is suffering from menorrhagia or a patient who is at risk from menorrhagia. Any premenopausal or perimenopausal woman is at risk of menorrhagia; however, menorrhagia is more common at the beginning and end of a woman's reproductive life so typically there is a greater risk when a woman's periods first start and in women over 40 years of age.

The patient to be treated may be any female individual who would benefit from such treatment. Typically and preferably the patient to be treated is a human female. However, the methods of the invention may be used to treat female mammals, such as the females of the following species: cows, horses, pigs, sheep, cats and dogs. Thus, the methods have uses in both human and veterinary medicine.

Typically, the agent is one which prevents or disrupts PGF_{2α}-mediated signalling of the FP receptor.

Preferably, an agent that prevents PGF_{2α} having its effect on the FP receptor prevents or reduces the binding of PGF_{2α} to the FP receptor. Alternatively or additionally, the agent may affect the interaction between PGF_{2α} and the FP receptor, or the interaction between the FP receptor and the associated G_{αq} protein, thus inhibiting or disrupting the PGF_{2α}-FP mediated signal transduction pathway.

In one preferred embodiment, the agent that prevents PGF_{2α} having its effect on the FP receptor may be an antagonist of the FP receptor. FP receptor antagonists are typically molecules which bind to the FP receptor, compete with the binding of the natural ligand PGF_{2α}, and inhibit or disrupt the PGF_{2α}-FP mediated signal transduction pathway. In one preferred embodiment, preventing PGF_{2α} having its effect on the FP receptor includes occupying the PGF_{2α} binding site on the prostaglandin receptor, such that the natural ligand (PGF_{2α}) is prevented from binding in a mode that would result in its normal mode of signalling via Gq/Gqπ through inositylphosphate and subsequent mobilisation of intracellular calcium.

Alternatively, the receptor antagonist may be a molecule which binds to the FP receptor without preventing PGF_{2α} binding thereto, but which disrupts the interaction between PGF_{2α} and the FP receptor, thus inhibiting or disrupting PGF_{2α}-FP mediated signal transduction pathway.

Further alternatively, the FP receptor antagonist may be a molecule which binds to the FP receptor and which disrupts the interaction between the FP receptor and the associated G_{αq} protein, thus inhibiting or disrupting FP mediated signal transduction pathway.

In an alternative preferred embodiment, the agent may be an antagonist of PGF_{2α}. PGF_{2α} antagonists are typically molecules which bind to PGF_{2α} and prevent or reduce PGF_{2α} binding to its receptor, which inhibits or disrupts the PGF_{2α}-FP mediated signal transduction pathway. This is the ‘soluble receptor’ approach in which typically either a part of the receptor or an antibody binds to PGF_{2α}.

Alternatively, the PGF_{2α} antagonist may be a molecule which binds to PGF_{2α} without preventing or reducing the binding of PGF_{2α} to the FP receptor, but which disrupts the interaction between PGF_{2α} and the FP receptor such that the PGF_{2α}-FP mediated signal transduction pathway is inhibited or disrupted. This could be a molecule which binds in a covalent fashion to PGF_{2α} and has no effect on binding potency but effects the G-protein/IP/Ca²⁺ mechanisms.

In one preferred embodiment, the agent that prevents PGF_{2α} having its effect on the FP receptor comprises an antagonist of the FP receptor, which may be any FP receptor antagonist that is suitable to be administered to a patient. The receptor antagonists are typically selective to the particular receptor and preferably have an equal or higher binding affinity to the FP receptor than does PGF_{2α}. Although antagonists with a higher affinity for the receptor than the natural ligand are preferred, antagonists with a lower affinity may also be used, but it may be necessary to use these at higher concentrations. Preferably, the antagonists bind reversibly to the FP receptor. Preferably, antagonists are selective for a particular receptor and do not affect other receptors; thus, typically, an FP receptor antagonist binds the FP receptor but does not substantially bind any other receptor.

The peptides listed in Table 1 are reported to be antagonists of the FP receptor that disrupt the interaction between the FP receptor and the associated G_{60 q} protein (WO 99/32640 and WO 00/17438). The amino acids are indicated according to the standard IUPAC single letter convention, and X is cyclohexyl alanine. Lower case letters indicate L-amino acids and capital letters indicate D-amino acids. All of the disclosure in WO 99/32640 and WO 00/17438 relating to specific peptides as FP receptor antagonists, is hereby incorporated herein by reference.

TABLE 1

| | |
|---|---|
| PCP-1 | RVKFKSQQHRQGRSHHLEM |
| PCP-2 | RKAVLKNLYKLASQCCGVHVISLHIWELSSIKNSLKVAAIS |
| | ESPVAEKSAST |
| PCP-3 | CLSEEAKEARRINDEIERQLRRDKRDARRE-NH₂ |
| PCP-4 | KDTILQLNLKEYNLV-NH₂ |
| PCP-8 | ilghrdyk |
| PCP-10 | wedrfyll |
| PCP-13 | ILGHRDYK |
| PCP-14 | YQDRFYLL |
| PCP-13.7 | ILAHRDYK |
| PCP-13.8 | ILaHRDYK |
| PCP-13.11 | ILGFRDYK |
| PCP-13.13 | ILGHKDYK |
| PCP-13.14 | ILGHRNYK |
| PCP-13.18 | ILGHQDYK |
| PCP-13.20 | ILGHRDY-amide |
| PCP-13.21 | ILGHRDYK-amide |
| PCP-13.22 | ILGWRDYK |
| PCP-13.24 | ILGXRDYK |
| PCP-15 | SNVLCSIF |

When the antagonist comprises a peptide, such as those mentioned in Table 1, the antagonist may also comprise protein fusions or peptidomimetics thereof.

PGF_{2α} dimethyl amide, obtained from Cayman Chemical, Ann Arbor, Mich., USA was reported to be a PGF_{2α} receptor antagonist (Amould et al., (2001) Am. J. Pathol., 159(1): 345-357).

U.S. Pat. No. 6,441,033 B1 (Sharif & Griffin, assigned to Alcon Manufacturing) describes 11β-fluoro 15β-hydroxy PGF_{2α} analogs which are FP receptor antagonists.

AL-8810 ((5Z, 13E)-(9S,11S,15R)-9,15-dihydroxy-11-fluoro-15-(2-indanyl)-16, 17, 18, 19, 20-pentanor-5,13-prostadienoic acid)obtained from Alcon Research was reported to be a weak partial agonist of the PGF_{2α} receptor and a highly selective antagonist of the PGF_{2α} receptor. AL-8810 was reported not to significantly inhibit functional responses of prostaglandin receptors TP, DP, EP2 or EP4 at high 10 μM concentration (Griffin et al., (1999) J. Pharmacol. Exp. Ther., 260(3): 1278-1284).

AL-3138 (11-deoxy-16-fluoro PGF_{2α}) was reported to be a weak partial agonist of the PGF_{2α} receptor, and also a highly selective antagonist of the PGF_{2α} receptor (Sharif et al., (2000) J. Pharm. Pharmacol., 52(12): 1529-1539).

Phloretin was reported to be a PGF_{2α} receptor antagonist (Kitanaka et al (1993) J. Neurochem. 60(2): 704-708).

The sulfonylurea glibenclamide was reported to be a PGF_{2α} receptor antagonist (Delaey and Van de Voorde (1995), Eur. J. Pharmacol. 280(2): 179-184). The sulfonylureas tolbutamide and tolazamide were reported to be very weak antagonists of the FP receptor. (Sharif et al (2000) J. Pharm. Pharmacol., 52(12): 1529-1539).

PGF_{2α} dimethyl amine was reported to be a PGF_{2α} receptor antagonist (Stinger et al (1992), J. Pharmacol. Exp. Ther., 220: 521-525).

(E)-5-[[[(3-pyridinyl)[3-(trifluoromethyl)phenyl]methylen]amino]oxy]pentanoic acid, also known as ridogrel, obtained from Jannsen Pharmaceutica, was reported to be a PGF_{2α} receptor antagonist (Jannsens et al., (1990), Thrombosis and Haemostasis, 64(1): 91-96).

The compound PHG113 was reported to be a selective PGF_{2α} receptor antagonist (Quiniou et al., (2001) Pediatric Research, 49(2): 452A.

EP-128479 describes pyrazolyl-methyl-ergoline derivatives which are reported to be PGF_{2α} receptor antagonists. All the disclosure in EP-128479 relating to pyrazolyl-methyl-ergoline derivatives as PGF_{2α} receptor inhibitors, is hereby incorporated herein by reference.

In a further preferred embodiment of the invention, the agent that prevents PGF_{2α} having its effect on the FP receptor may be an antagonist of PGF_{2α}, which may be any PGF_{2α} antagonist that is suitable to be administered to the patient. The PGF_{2α} antagonists are preferably selective to PGF_{2α} and typically have a higher binding affinity for PGF_{2α} than for other molecules. Although antagonists with a higher affinity for PGF_{2α} than other molecules are preferred, antagonists with a lower affinity may also be used, but it may be necessary to use these at higher concentrations. Preferably, the PGF_{2α} antagonists bind reversibly to PGF_{2α}.

PGF_{2α} antagonists include anti-PGF_{2α} antibodies such as rabbit polyclonal anti-PGF_{2α} antibodies from Oxford Biomedical Research, Inc., Oxford, UK (Arnould et al., Am. J. Pathol. 2001 159(1): 345-357). Amould et al state that, according to the manufacturer, the specificity of the antibody is very high and the cross-reactivity with other prostanoid derivatives is <1%.

JP 04077480; JP 08176134; JP 01199958; JP 01050818; and JP 63083081 each describe phthalide derivatives that are reported to be PGF_{2α} inhibitors. All the disclosure in JP 04077480; JP 08176134; JP 01199958; JP 01050818; and JP 63083081 relating to phthalide derivatives as PGF_{2α} inhibitors, is hereby incorporated herein by reference.

WO 91/13875 describes (iso) quinoline sulphonamide compounds which are reported to be PGF_{2α} inhibitors. All the disclosure in WO 91/13875 relating to (iso) quinoline sulphonamide compounds as PGF_{2α} inhibitors, is hereby incorporated herein by reference.

Some of the compounds reported as being inhibitors or antagonists of PGF_{2α} may, in fact, be antagonists of the PGF_{2α} (FP) receptor, as used and defined herein. References to such compounds as inhibitors or antagonists of PGF_{2α} should therefore be considered as references to FP receptor antagonists.

As used herein, the term ‘antagonist’ covers all types of antagonism. GPCRs such as prostaglandin receptors are known to show inverse agonism which has the outcome of blocking a desired response. Thus a suitable FP antagonist for use in the present invention may be identified by measuring the binding of a radio-labelled FP agonist to PGF_{2α} with or without the purported antagonist. Secondly, FP antagonists may be identified in a functional assay eg by showing that the effect of an FP agonist on Ca²⁺ levels is modified in the presence of the antagonist. Thirdly FP antagonists may be identified by inhibition of epithelial cell growth in cell culture.

All of the patents and other documents referred to herein, and in particular those describing antagonists or inhibitors of FP receptors and of PGF_{2α}, are incorporated herein, in their entirety, by reference.

We have previously found elevated expression of EP2 and EP4 receptors in the endometrium of menorrhagic women compared to control women (see Example 3 and PCT/GB02/04845) and have suggested the use of an inhibitor of PGES or an EP2 or EP4 receptor antagonist in the treatment or prevention of menorrhagia (PCT/GB02/004845).

Thus, in a further embodiment of the present invention, in addition to the at least one agent that prevents PGF_{2α} having its effect on the FP receptor, the individual is also administered one or more of an inhibitor of PGES and/or an antagonist of EP2 or of EP4.

In one embodiment of the invention, the individual is administered an inhibitor of PGES. It has been reported by Thoren & Jakobsson (2000) Eur. J Biochem. 267, 6428-6434 (incorporated herein by reference) that NS-398, sulindac sulphide and leukotriene C₄ inhibit PGES activity with IC₅₀ values of 20 μM, 80 μM and 5 μM, respectively.

In a still further embodiment of the invention, the individual is administered an antagonist of an EP2 receptor or an antagonist of an EP4 receptor. It will be appreciated that an antagonist of an EP2 receptor or an antagonist of an EP4 receptor is an agent that prevents PGE₂ having its effect on the said EP2 or EP4 receptor.

The prostaglandin EP2 receptor antagonist may be any suitable EP2 receptor antagonist. Similarly, the prostaglandin EP4 receptor antagonist may be any suitable EP4 receptor antagonist. By “suitable” we mean that the antagonist is one which may be administered to a patient. The receptor antagonists are molecules which bind to their respective receptors, compete with the natural ligand (PGE₂) and inhibit the initiation of the specific receptor-mediated signal transduction pathways. The receptor antagonists are typically selective to the particular receptor and typically have a higher binding affinity to the receptor than the natural ligand. Although antagonists with a higher affinity for the receptor than the natural ligand are preferred, antagonists with a lower affinity may also be used, but it may be necessary to use these at higher concentrations. Preferably, the antagonists bind reversibly to their cognate receptor. Typically, antagonists are selective for a particular receptor and do not affect the other receptor; thus, typically, an EP2 receptor antagonist binds the EP2 receptor but does not substantially bind the EP4 receptor, whereas an EP4 receptor antagonist binds the EP4 receptor but does not substantially bind the EP2 receptor. Preferably, the EP2 or EP4 receptor antagonist is selective for the particular receptor subtype. By this is meant that the antagonist has a binding affinity for the particular receptor subtype which is at least ten-fold higher than for at least one of the other EP receptor subtypes. Thus, selective EP4 receptor antagonists have at least a ten-fold higher affinity for the EP4 receptor than any of the EP1, EP2 or EP3 receptor subtypes.

It is particularly preferred that the EP2 or EP4 receptor antagonist is selective for its cognate receptor.

EP2 receptor antagonists include AH6809 (Pelletier et al (2001) Br. J. Pharmacol. 132, 999-1008).

EP4 receptor antagonists include AH23848B (developed by Glaxo) and AH22921X (Pelletier et al (2001) Br. J. Pharmacol. 132, 999-1008. The chemical name for AH23848B is ([1alpha(z), 2beta5alpha]-(±)-7-[5-[[(1,1′-biphenyl)-4-yl]methoxy]-2-(4-morpholinyl)-3-oxo-cyclopentyl]-4-heptenoic acid) (see Hillock & Crankshaw (1999) Eur. J. Pharmacol. 28, 99-108). EP4RA (Li (2000) Endocrinology 141, 2054-61) is an EP(4)-selective ligand (Machwate et al (2001) Mol. Pharmacol. 60: 36-41). The omega-substituted 5 prostaglandin E derivatives described in WO 00/15608 (EP 1 114 816) (Ono Pharm Co Ltd) bind EP4 receptors selectively and may be EP4 receptor antagonists.

Peptides described in WO 01/42281 (Hopital Sainte-Justine) eg: IFTSYLECL, IFASYECL, IFTSAECL, IFTSYEAL, ILASYECL, IFRSTDCL, TSYEAL (with 4-biphenyl alanine), TSYEAL (with homophenyl alanine) are also described as EP4 receptor antagonists, as are some of the compounds described in WO 00/18744 (Fujisawa Pharm Co Ltd). The 5-thia-prostaglandin E derivatives described in WO 00/03980 (EP 1 097 922) (Ono Pharm Co Ltd) may be EP4 receptor antagonists.

EP4 receptor antagonists are also described in WO 01/10426 (Glaxo), WO 00/21532 (Merck) and GB 2 330 307 (Glaxo).

WO 00/21532 describes the following as EP4 receptor antagonists:

5-butyl-2,4-dihydro-4-[[2′-[N-(3-chloro-2-thiophenecarbonyl)sulfamoyl]biphenyl-4-yl]methyl]-2-{2-(trifluoromethyl)phenyl]-1,2,4-triazol-3-one potassium salt;

5-butyl-2,4-dihydro-4-[[2′-[N-(2-methyl-3-furoyl)sulfamoyl]biphenyl-4-yl]methyl]-2-{2-(trifluoromethyl)phenyl]-1,2,4-triazol-3-one;

5-butyl-2,4-dihydro-4-[[2′-[N-(3-methyl-2-thiophenecarbonyl)sulfamoyl]biphenyl-4-yl]methyl]-2-{2-(trifluoromethyl)phenyl]-1,2,4-triazol-3-one;

5-butyl-2,4-dihydro-4-[[2′-[N-(2-thiophenecarbonyl)sulfamoyl]biphenyl-4-yl]methyl]-2-{2-(trifluoromethyl)phenyl]-1,2,4-triazol-3-one;

5-butyl-2,4-dihydro4-[[2′-[N-[2-(methypyrrole)carbonyl]sulfamoyl]biphenyl-4-yl]methyl]-2-{2-(trifluoromethyl)phenyl]-1,2,4-triazol-3-one.

GB 2 330 307 describes [1α(Z), 2β, 5α9 -(±)-7-[5-[[(1,1′-biphenyl)-4-yl]methoxy]-2-(4-morpholinyl)-3-oxocyclopentyl]-4-heptenoic acid and [1R[1α(z),2β,5α]]-(−)-7-[5-[[(1,1′-biphenyl)-4-yl]methoxy]-2-(4-morpholinyl)-3-oxocyclopentyl]-4-heptenoic acid.

WO 00/18405 (Pharmagene) describes the EP4 receptor antagonists AH22921 and AH23848 (which are also described in GB 2 028 805 and U.S. Pat. No. 4,342,756). WO 01/72302 (Pharmagene) describes further EP4 receptor antagonists, for example those described by reference to, and included in the general formula (I) shown on page 8 et seq.

In an embodiment, when one or more of an inhibitor of PGES and/or an antagonist of EP2 or of EP4 is administered to a patient, in addition to the at least one agent that prevents PGF_{2α} having its effect on the FP receptor, the dose of each compound may be the same as would be administered individually without reference to the other compound. Alternatively and preferably, lower doses may be administered.

All of the patents and other documents referred to herein that describe antagonists or inhibitors of EP2 or EP4 and PGES, are incorporated herein, in their entirety, by reference.

It will be appreciated that one or more agents that prevent PGF_{2α} having its effect on the FP receptor may be administered to the patient. Optionally, one or more of an inhibitor of PGES and/or an antagonist of EP2 or of EP4 may also be administered to the patient. These may all be considered “treatment agents” of the invention. It will also be appreciated that when more than one treatment agent is administered to the patient, they may be administered sequentially or in combination.

The treatment agent(s) are administered in an effective amount to combat the menorrhagia. Thus, the treatment agents may be used to alleviate symptoms (ie are used palliatively), or may be used to treat the condition, or may be used prophylactically to prevent the condition. The treatment agent may be administered by any suitable route, and in any suitable form.

Typically, the aforementioned treatment agent(s) for use in the invention is administered in a quantity and frequency such that an effective dose is delivered to at least 90% of the FP, and optionally the EP2 and/or EP4, receptors (ED₉₀). The potency of the molecule(s) would dictate the dose, as would the formulation and route of administration.

The aforementioned treatment agents for use in the invention or a formulation thereof may be administered by any conventional method including oral and parenteral (eg subcutaneous or intramuscular) injection. The treatment may consist of a single dose or a plurality of doses over a period of time. The dose to be administered is determined upon consideration of age, body weight, mode of administration, duration of the treatment and pharmacokinetic and toxicological properties of the treatment agent or agents. The treatment agents are administered at a dose (or in multiple doses) which produces a beneficial therapeutic effect in the patient. Typically, the treatment agents are administered at a dose the same as or similar to that used when the treatment agent is used for another medical indication. In any event, the dose suitable for treatment of a patient may be determined by the physician.

Whilst it is possible for a treatment agent of the invention to be administered alone or in combination with other said treatment agents, it is preferable to present it or them as a pharmaceutical formulation, together with one or more acceptable carriers. The carrier(s) must be “acceptable” in the sense of being compatible with the treatment agent of the invention and not deleterious to the recipients thereof. Typically, the carriers will be water or saline which will be sterile and pyrogen free.

The formulations may conveniently be presented in unit dosage form and may be prepared by any of the methods well known in the art of pharmacy. Such methods include the step of bringing into association the treatment agent or agents with the carrier which constitutes one or more accessory ingredients. In general the formulations are prepared by uniformly and intimately bringing into association the active ingredient (ie treatment agent or agents) with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier. Buccal administration is also preferred.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

Certain of the treatment agents are proteins or peptides. Proteins and peptides may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The protein and peptide can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy (EPT) systems can also be employed for the administration of proteins and peptides. A device which delivers a pulsed electric field to cells increases the permeability of the cell membranes to the drug, resulting in a significant enhancement of intracellular drug delivery.

Proteins and peptides can be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of protein and peptide delivery is the ReGel injectable system that is thermo-sensitive. Below body temperature, ReGel is an injectable liquid while at body temperature it immediately forms a gel reservoir that slowly erodes and dissolves into known, safe, biodegradable polymers. The treatment agent is delivered over time as the biopolymers dissolve.

Protein and peptide pharmaceuticals can also be delivered orally. The process employs a natural process for oral uptake of vitamin B₁₂ in the body to co-deliver proteins and peptides. By riding the vitamin B₁₂ uptake system, the protein or peptide can move through the intestinal wall. Complexes are synthesised between vitamin B₁₂ analogues and the drug that retain both significant affinity for intrinsic factor (IF) in the vitamin B₁₂ portion of the complex and significant bioactivity of the drug portion of the complex.

Proteins and polypeptides can be introduced to cells by “Trojan peptides”. These are a class of polypeptides called penetratins which have translocating properties and are capable of carrying hydrophilic compounds across the plasma membrane. This system allows direct targeting of oligopeptides to the cytoplasm and nucleus, and may be non-cell type specific and highly efficient. See Derossi et al (1998), Trends Cell Biol 8, 84-87.

The treatment agents or formulations may also be administered transdermally, eg as a patch, gel, lotion, cream or oil.

It is preferred if the treatment agent (or agents) is administered orally.

It is further preferred if the treatment agent (or agents) is administered to the female reproductive system. For example, the treatment agent or agents may suitably be administered intravaginally using, for example, a gel or cream or vaginal ring or tampon. The treatment agent may also advantageously be administered by intrauterine delivery, for example using methods well known in the art such as an intrauterine device.

Typically, the gel or cream is one which is formulated for administration to the vagina. It may be oil based or water based. Typically, the treatment agent (or agents) is present in the cream or gel in a sufficient concentration so that an effective amount is administered in a single (or in repeated) application.

Typically, the vaginal ring comprises a polymer which formed into a “doughnut” shape which fits within the vagina. The treatment agent (or agents) is present within the polymer, typically as a core, which may dissipate through the polymer and into the vagina and/or cervix in a controlled fashion. Vaginal rings are known in the art. The vaginal ring may be disposable and is retained intravaginally during the woman's period and therefore contains sufficient of the treatment agent(s) to be released and to be effective during the woman's period. Alternatively, the vaginal ring may be used over a time interval of around three months to one year, during which time sufficient of the treatment agent(s) is released to have a beneficial effect over that period of time. It will be appreciated that the polymer from which the ring is made, the size and shape of the ring and the content of the treatment agent, as well as other parameters, may be selected by reference to whether the ring is for use in one cycle or for longer spells.

Typically, the tampon is impregnated with the treatment agent (or agents) and that a sufficient amount of the treatment agent (or agents) is present in the tampon.

Typically, the intrauterine device is for placing in the uterus over extended periods of time, such as between one and five years. Typically, the intrauterine device comprises a plastic frame, often in the shape of a “T” and contains sufficient of the treatment agent(s) to be released over the period of use. The agent is generally present within or encompassed by a slow-release polymer which forms part of the device, such as in the form of a “sausage” of agent which wraps around the long arm of the “T” which is typically covered with a controlled-release membrane. Intrauterine devices are known in the art.

The invention also provides combinations (such as in a pharmaceutical formulation) of one or more of the treatment agents as described herein, and one or more agents presently used to treat menorrhagia, such as tranexamic acid or mefenamic acid.

A second aspect of the invention provides use of at least one agent that prevents PGF_{2α} having its effect on the FP receptor, in the manufacture of a medicament for treating or preventing menorrhagia in a female individual.

It is appreciated that in this and all subsequent aspects of the invention, preferences for an agent that prevents PGF_{2α} having its effect on the FP receptor are as described previously with respect to the first aspect of the invention.

In an embodiment, the female individual is administered one or more of an inhibitor of PGES and/or an antagonist of EP2 or EP4. Typically the female is administered the one or more of these additional agents at the same time as the medicament. Alternatively, the female may have been administered the one or more of these additional agents before receiving the medicament containing the at least one agent that prevents PGF_{2α} having its effect on the FP receptor. Further alternatively, the female will be administered the one or more of these additional agents after receiving the medicament containing the at least one agent that prevents PGF_{2α} having its effect on the FP receptor.

It is appreciated that in this and all subsequent aspects of the invention, preferences for an inhibitor of PGES and/or an antagonist of EP2 or EP4 are as described previously with respect to the first aspect of the invention.

A third aspect of the invention provides use of a combination of at least one agent that prevents PGF_{2α} having its effect on the FP receptor, and one or more of an inhibitor of PGES and/or an antagonist of EP2 or EP4, in the manufacture of a medicament for treating or preventing menorrhagia in a female individual.

A fourth aspect of the invention provides use of one or more of an inhibitor of PGES and/or an antagonist of EP2 or EP4, in the manufacture of a medicament for treating or preventing a pathological condition of the uterus in a female individual, wherein the individual is administered at least one agent that prevents PGF_{2α} having its effect on the FP receptor. In this case, typically the female is administered the at least one agent that prevents PGF_{2α} having its effect on the FP receptor at the same time as the medicament, although the female may have been (or will be) administered the at least one agent that prevents PGF_{2α} having its effect on the FP receptor before (or after) receiving the medicament.

A fifth aspect of the invention provides a pharmaceutical composition comprising at least one agent that prevents PGF_{2α} having its effect on the FP receptor, for treating or preventing menorrhagia.

Optionally, the pharmaceutical composition further comprises one or more of an inhibitor of PGES and/or an antagonist of EP2 or EP4.

It is believed that there has been no previous description of a composition comprising at least one agent that prevents PGF_{2α} having its effect on the FP receptor, and an inhibitor of PGES and/or an antagonist of EP2 or EP4. Furthermore, it is believed that there has been no previous suggestion that such a composition could be used to treat any medical condition.

Therefore, in a further aspect, the invention includes a composition comprising at least one agent that prevents PGF_{2α} having its effect on the FP receptor, and one or more of an inhibitor of PGES and/or an antagonist of EP2 or EP4.

In a yet further aspect, the invention includes a pharmaceutical composition comprising at least one agent that prevents PGF_{2α} having its effect on the FP receptor, and one or more of an inhibitor of PGES and/or an antagonist of EP2 or EP4, and a pharmaceutically acceptable carrier.

In a still further aspect, the invention includes a pharmaceutical composition comprising at least one agent that prevents PGF_{2α} having its effect on the FP receptor, and one or more of an inhibitor of PGES and/or an antagonist of EP2 or EP4, for use in medicine.

Thus the composition is packaged and presented for use in medicine.

The invention will now be described in more detail by reference to the following Figures and Examples.

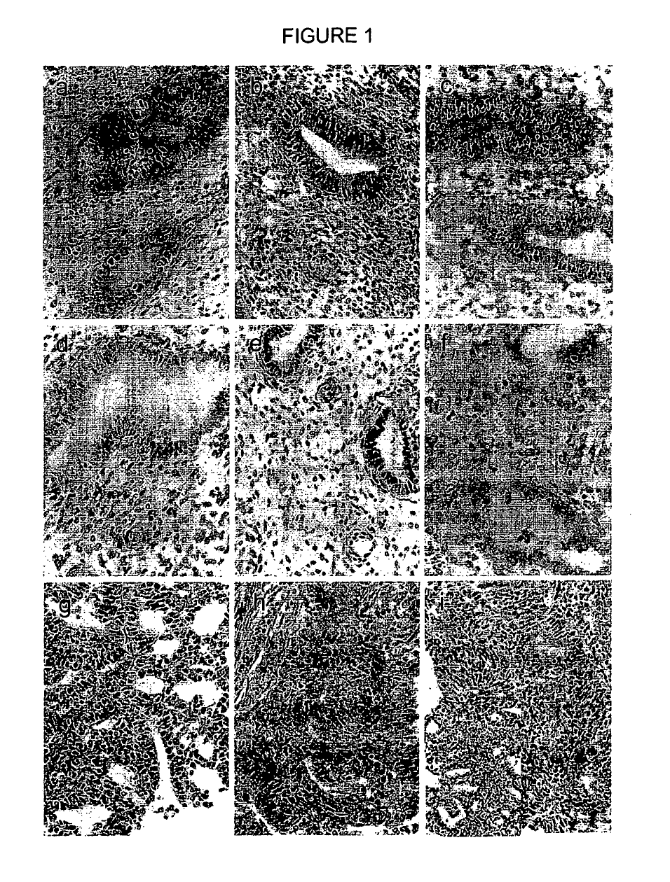

FIG. 1

In situ hybridisation for FP receptor. Panels a-f are normal human endometrium in the early-proliferative phase (a), mid-proliferative phase (b), late-proliferative phase (c), early-secretory phase (d), mid-secretory phase (e) and late-secretory phase (f). Panels g-i are poor (g), moderate (h) and well (i) differentiated samples of adenocarcinoma tissue.

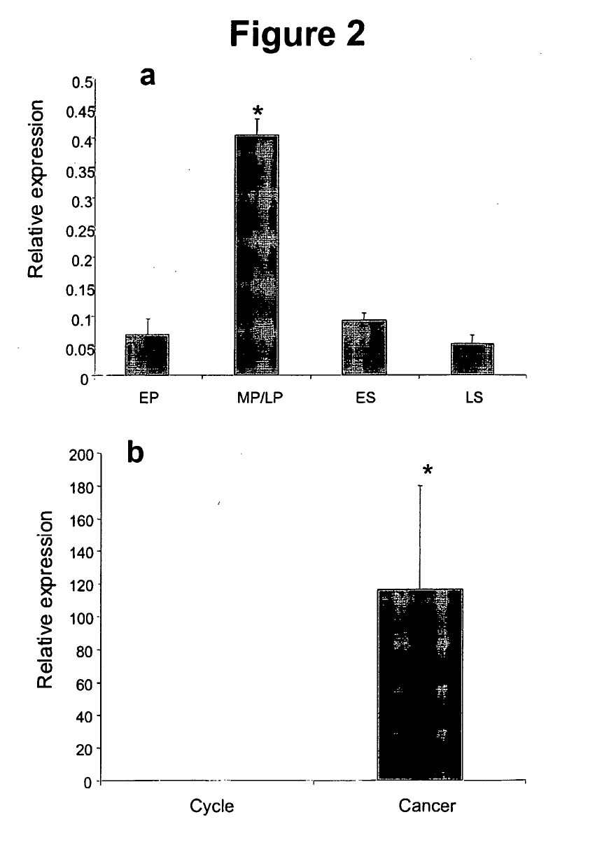

FIG. 2

Quantitative RT-PCR expression of FP receptor in a) normal human endometrium and b) adenocarcinoma tissues (p<0.05). In a), EP is early proliferative phase, MP/LP is mid/late proliferative phase, ES is early secretory phase and LS is late secretory phase. In b), “Cycle” is an average of EP, MP/LP, ES and LS from a).

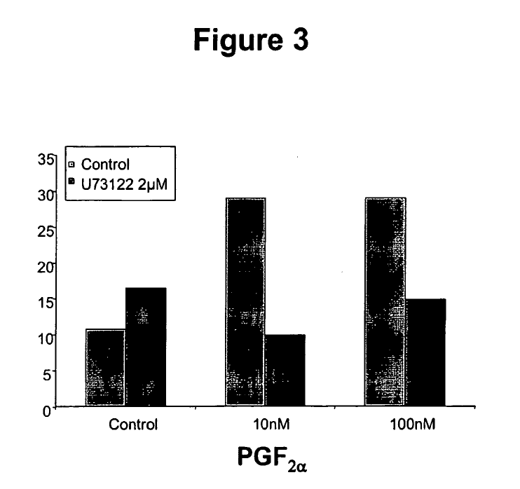

FIG. 3

Total inositol phosphate production in human endometrium (n=2).

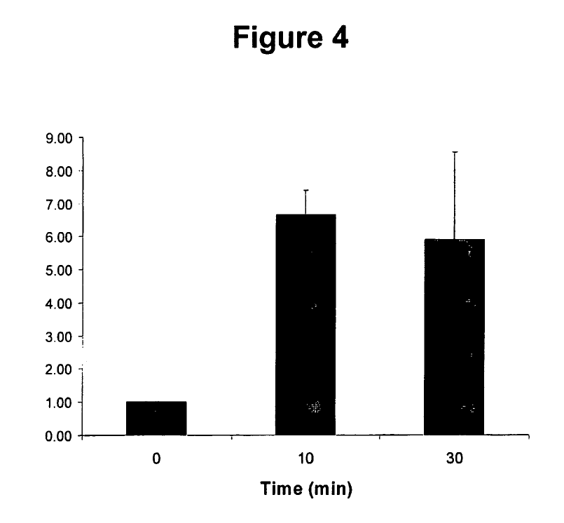

FIG. 4

PGF_{2α}-induced ERK phosphorylation in Ishikawa cells. PGF_{2α} 100 nM was incubated for 10 and 30 min. U73122 2 μM was pre-incubated for 60 min before treatment with PGF_{2α}.

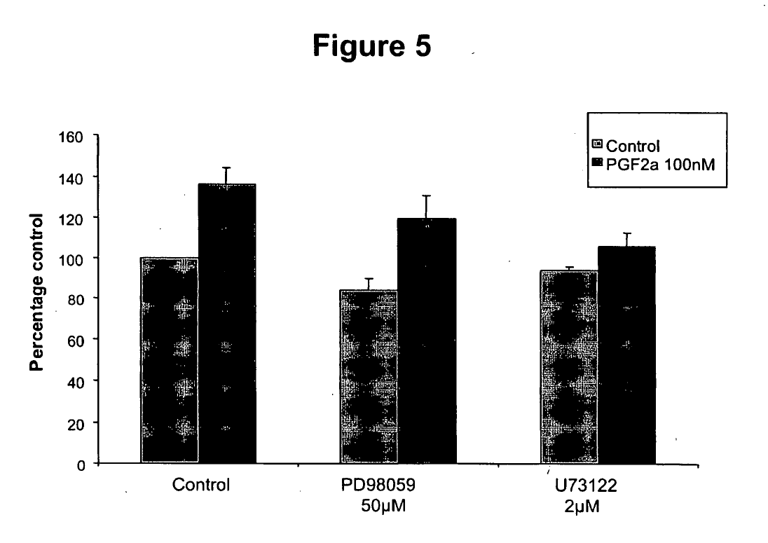

FIG. 5

PGF_{2α}-induced BrdU incorporation. Proliferation in Ishikawa cells following PGF_{2α}. Cells were treated overnight with PGF_{2α}. All inhibitors were added for 60 min before the addition of PGF_{2α} (n≧4; p<0.05).

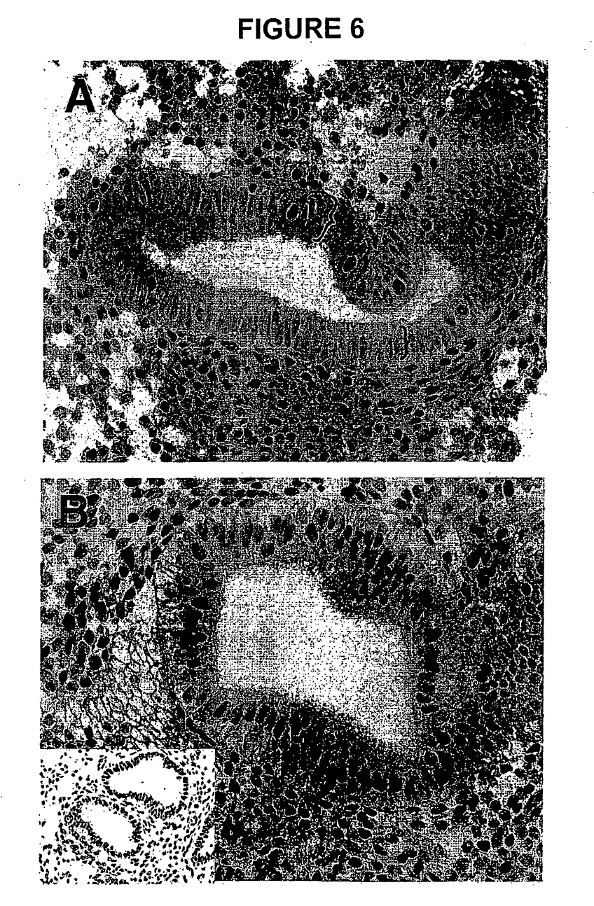

FIG. 6

Inmmunocytochemistry of FP receptor expression in human endometrium collected from women with excessive (A) or normal (B) blood loss. The inset in B is a negative control. The endometrial tissue from both menorrhagic and normal women was collected during the secretory phase of the menstrual cycle. In (A), the region of menorrhagic tissue which stained for expression of the FP receptor is indicated by“}”.

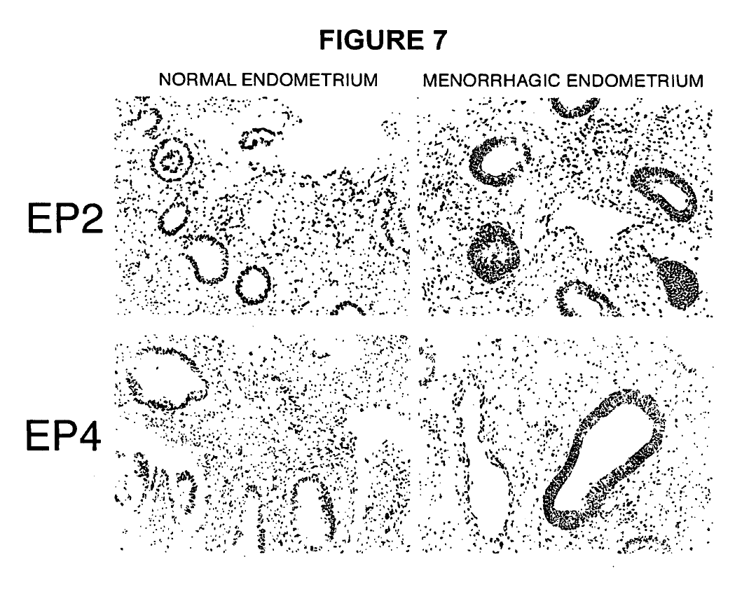

FIG. 7

Endometrial sections from menorrhagic and control women stained with antibodies to the EP2 receptor and EP4 receptor.

### EXAMPLE 1

### Prostaglandin (PG) F_{2α} Receptor Expression and Localisation in Human Endometrium and Endometrial Adenocarcinoma: Role of PGF_{2α} in Epithelial Cell Proliferation

Summary

PGF_{2α} is one of the prostaglandins generated by human endometrium and can be measured in menstrual fluid and from endometrial explants cultured in vitro. PGF_{2α} production is stimulated by oestrogen, which induces an up-regulation of COX expression, and inhibited by progesterone, which decreases COX expression and increases PGDH expression. Increased COX expression has been demonstrated in a number of different cancers and over-expression in rat intestinal epithelial (RIE) cells induces an altered cell type with increased proliferation and invasiveness in vivo. The aims of this study were to identify the target cells for PGF_{2α} in human endometrium and endometrial adenocarcinoma and quantify FP receptor expression in these tissues. The role of PGF_{2α} in epithelial cell proliferation, and the specific signalling pathways involved, was then determined in an endometrial adenocarcinoma cell line (Ishikawa). We observed a significantly increased expression of FP mRNA in mid- to late-proliferative tissue that was further increased in endometrial adenocarcinomas. Localisation of FP mRNA was identified in epithelial cells from only mid- and late-proliferative tissue samples. In uterine adenocarcinoma tissue, FP expression was consistently localised in epithelial cells and was independent of differentiation stage. Negligible FP mRNA expression was seen in adjacent stromal cells. Inositol mobilisation in response to PGF_{2α} was determined in normal endometrium and endometrial adenocarcinoma and was significantly increased in all samples following PGF_{2α}, however, no additional inositol mobilisation was found in adenocarcinoma samples. In Ishikawa cells, PGF_{2α} also induced inositol mobilisation and produced a concentration-dependent increase in cell proliferation that was inhibited PLC-dependent but not affected by either p38 or p42/p44 MAPK inhibitors. These results demonstrate that proliferating endometrial epithelial cells are responsive to PGF_{2α} and indicate a role for PGF_{2α} in menorrhagia.

Introduction

Prostaglandin (PG) F_{2α} is a prostanoid belonging to the eicosanoid family of biologically active lipid (1). Other members of this prostanoid family include PGD₂, PGE₂, prostacyclin (PGI₂) and thromboxane A₂ (TxA₂) that are all synthesised from arachidonic acid by a combination of cyclooxygenase (COX) and specific synthase enzymes (2). To-date, there are two identified isoforms of the COX enzyme; constitutively expressed COX-1 that generates PG for normal physiological function and COX-2, an early response gene whose expression can be rapidly induced (3). COX metabolises arachidonic acid to the unstable intermediate PGH₂ and specific synthase enzymes then convert PGH₂ to the PG molecules (4-8). The PG synthase enzymes are named according to the prostaglandin they produce such that PGF_{2α} is a metabolite of prostaglandin-F-synthase. Once synthesised PG mediate their actions via seven-transmembrane G-protein coupled receptors (GPCR) specific to each prostanoid. PGF_{2α} receptor (FP) has been cloned in humans and transduces its response via the G-protein Gq, PLC activation and generation of inositol-trisphosphate that in turn mobilises intracellular Ca²⁺.

COX enzymes and PG have recently been demonstrated to regulate epithelial cell growth and angiogenesis. In rat intestinal epithelial cells, COX-2 expression and PGE₂ synthesis are associated with increased cellular proliferation and resistance to apoptosis (9,10). The same group also showed that expression of COX-2 in epithelial cells enhances the expression of angiogenic factors that act in a paracrine manner to induce endothelial cell migration and microvascular tube formation (9). In human endometrium, COX-2 enzyme expression is maximal during the proliferative phase and is localised to epithelial and perivascular cells (10-14).

PGF_{2α} is also a major metabolite of COX in human endometrium and as well as being present in menstrual fluid is released by human endometrial explants in culture (15). However, due to the potent luteolytic activity of PGF_{2α} and the demonstrated increase in myometrial contractility following PGF_{2α}, most studies have focused on FP receptor expression and regulation in the ovary. FP receptor expression and the role of PGF_{2α} within the functionalis layer of human endometrium have not been fully examined.

Original studies identified in separated bovine endometrial cells cultured in vitro, that epithelial cells preferentially release PGF_{2α} in contrast to stromal cells that secrete predominately PGE2 (16). Moreover, studies measuring FP mRNA in non-primate species demonstrated increased FP receptor expression with oestrogen where as progesterone decreased FP receptor expression when animal were ovariectomised (17-19).

The aims of this study were to localise and quantify FP receptor expression both in human endometrium and in differing grades of human uterine adenocarcinoma to identify the PGF_{2α}-responsive cells. Mobilisation of inositol phosphates and phosphorylation of the MAPK extracellular-regulated kinase (ERK) were used to determine the presence of functional FP receptors and BrdU incorporation in Ishikawa cells as an in-vitro proliferation assay. The data from this study demonstrate epithelial expression of FP in only proliferating tissue with substantially increased FP mRNA and clear epithelial cell localisation in endometrial adenocarcinomas. Moreover, PGF_{2α} treatment of Ishikawa cells caused inositol mobilisation, ERK phosphorylation and concentration-dependent increases in cell proliferation.

The data reported herein is the first report of FP expression in human endometrium and demonstrates a potential role of PGF_{2α} in uterine epithelial cell proliferation and in menorrhagia.

Methods

Patients and Tissue Collection

Endometrial biopsies (n=12) at different stages of the menstrual cycle were collected with an endometrial suction curette (Pipelle, Laboratoire CCD, France) from women with regular menstrual cycles (25-35 days). In addition, full thickness endometrial biopsies (n=18) at all stages of the menstrual cycle (n=3 from early, mid and late proliferative and n=3 from early mid and late secretory) were collected from women undergoing hysterectomy for benign gynaecological indications. Shortly after pipelle suction or hysterectomy, tissue was either snap frozen in dry ice and stored at −70° C. (for RNA extraction), fixed in neutral buffered formalin (NBF) and wax embedded (for in-situ hybridisation studies), or placed in RPMI 1640 (containing 2 mmol/l L-glutamine, 100 U penicillin and 100 μg/ml streptomycin) and transported to the laboratory for in vitro culture. All subjects reported regular menstrual cycles (cycle length 25-35 days) and no women had received a hormonal preparation in the 3 months preceding biopsy collection. Biopsies were dated according to stated last menstrual period (LMP) and confirmed by histological assessment according to criteria of Noyes and co-workers (20). Furthermore, circulating oestradiol and progesterone concentrations at the time of biopsy were consistent for both stated LMP and histological assignment of menstrual cycle stage. Ethical approval was obtained from Lothian Research Ethics Committee and written informed consent was obtained from all subjects before tissue collection.

In situ Hybridisation (ISH)

Custom synthesis oligonucleotide double FITC-labelled cDNA probes for FP receptor were obtained from Biognostik GmbH (Germany). Sections (5 μm) were cut onto Gelatin coated Superfrost slides (BDH Laboratory Supplies, UK) from full thickness human uterine biopsies collected across the menstrual cycle (n=18). Tissue was dewaxed in xylene, rehydrated using increasing concentrations of ethanol before Proteinase K treatment (100 μg/ml in Tris-HCl pH 7.6 100 mM containing EDTA 50 mM) for 15 min at 37° C. to enhance cDNA probe access. After washing in DEPC-H₂O, hybridisation mixture (50 μl; supplied with probe) was added to each section and slides incubated for 4 h at 30° C. before adding cDNA probe (6U/ml hybridisation mix) and incubating overnight at 30° C. Post-hybridisation washes of 1×SSC for 5 min (twice) and 0.1×SSC at 42° C. for 15 min (twice) were completed before detecting the FITC-labelled probe using standard ICC reagents (TSA Biotin System, NEN Life Sciences, UK). Endogenous peroxidase activity was first blocked with 3% H₂O₂ in methanol for 30 min before incubating sections with blocking buffer for 30 min. Conjugated anti-FITC-HRP (Boehringer-Mannheim, Check) was added in blocking buffer and the sections incubated for 60 min. After washing, biotinyl tyramide amplification reagent was applied to each slide and incubated for 15 min. Streptavidin-HRP was applied after washing and incubated for 30 min and probe localisation visualised with DAB. Control oligonucleotide double FITC-labelled cDNA probe containing the same proportion of cysteine (C) and guanine (G) bases as the FP receptor probe was included to assess background hybridisation. All treatments were carried out at room temperature unless otherwise specified.

Taqman Quantitative RT-PCR

Endometrial RNA samples were extracted from endometrial biopsies (n=33) using Tri-reagent (Sigma, UTK) following the manufacturer's guidelines. Once extracted and quantified, RNA samples were reverse transcribed using MgCl₂ (5.5 mM), dNTPs (0.5 mM each), random hexamers (2.5 μM), RNAase inhibitor (0.4 U/μl) and multiscribe reverse transcriptase (1.25 U/μl; all from PE Biosystems, Warrington, UK). The mix was aliquoted into individual tubes (16 μl/tube) and template RNA was added (4 μl/tube of 100 ng/μl RNA). After mixing by brief centrifugation, samples were incubated for 90 minutes at 25° C., 45 minutes at 48° C. and 95° C. for 5 minutes. Thereafter cDNA samples were stored at −20° C. A tube with no reverse transcriptase was included to control for any DNA contamination.

To measure cDNA expression a reaction mix was prepared containing Taqman buffer (5.5 mM MgCl₂, 200 μM dCATP, 200 μM dCTP, 200 μM dGTP, 400 μM dUTP), ribosomal 18S forward and reverse primers and probe (all at 50 nM), forward and reverse primers for EP receptor (300 nM), EP receptor probe (100 nM), AmpErase UNG (0.01 U/μl) and AmpliTaq Gold DNA Polymerase (0.025 U/μl; all from PE Biosystems). After mixing, 48 μl was aliquoted into separate tubes and 2 μl/replicate (40 ng) of cDNA added and mixed before placing duplicate 24 μl samples into a PCR plate. A no template control (containing water) was included in triplicate. Wells were sealed with optical caps and the PCR reaction carried out using an ABI Prism 7700. FP receptor primers and probe for quantitative PCR were designed using the PRIMER express program (PE Biosystems). The sequence of the FP receptor primers and probe were; Forward 5′-GCA GCT GCG CTT CTT TCA A-3′; Reverse 5′-CAC TGT CAT GAA GAT TAC TGA AAA AAA TAC-3′; Probe (FAM labelled) 5′-CAC AAC CTG CCA GAC GGA AAA CCG-3′.

The ribosomal 18S primers and probe sequences were; Forward 5′-CGG CTA CCA CAT CCA AGG AA-3′; Reverse 5′-GCT GGA ATT ACC GCG GCT-3′; Probe (VIC labelled) 5′-TGC TGG CAC CAG ACT TGC CCT C-3′. Data were analysed and processed using Sequence Detector vl.6.3 (PE Biosystems) as instructed by the manufacturer. Briefly, the software calculates the reaction cycle number at which fluorescence reaches a determined level for both 18S control and FP receptor. This is the relative abundance of FP receptor in each sample and by comparing to an internal positive control, relative expression can be determined. Results are expressed as relative expression to the internal positive standard.

Total Inositol Phosphate (InsP) Assays

PGF_{2α} stimulation of total InsP production was as described (21). Briefly, tissue samples or Ishikawa cells were incubated with inositol free DMEM containing 1% dialyzed heat-inactivated FCS and 0.5 μCi/well myo-[³H]inositol (Amersham Pharmacia Biotech) for 48 hours. Medium was removed, and cells washed with 1 ml buffer (140 mM NaCl, 20 mM HEPES, 4 mM KCl, 8 mM glucose, 1 mM MgCl₂, 1 mM CaCl₂, 1 mg/ml bovine serum albumin) containing 10 mM LiCl. Cells were then incubated for 1 hour at 37° C. in 1 ml buffer with or without inhibitors. Following incubation agonist was added at the required concentration and cells incubated for 1 hour. Reactions were terminated by the removal of agonist and the addition of 500 μl ice cold 10 mM formic acid, which was incubated for 30 minutes at 4° C. Total [³H] inositol phosphates was separated from the formic acid cell extracts on AG 1-X8 anion exchange resin (Bio-Rad) and eluted with a 1 M ammonium formate/0.1 M formic acid solution. The associated radioactivity was determined by liquid scintillation counting and plotted relative to protein concentrations determined using the modified Lowry method.

Proliferation Assay

Proliferation of Ishikawa cells was determined using a BrdU incorporation ELISA (Roche Diagnostics GmbH, Mannheim, Germany). Briefly, Ishikawa cells were seeded at 5×10³ cells per well in a 96-well plate and allowed to adhere overnight. Cells were next starved for 24 hr with indomethacin 1.5 μg/ml before 24 h PGF_{2α} treatment (1 nM-1 μM) in serum-free medium containing indomethacin. Inhibitors were added to cells for 60 min before PGF_{2α} with the control well receiving the same concentration of vehicle. Following 24 h treatment, cells were labelled with BrdU for 4 h, then fixed and assayed for BrdU incorporation using standard immunohistochemical techniques. Results were plotted as percentages of untreated cells.

Statistics

Where appropriate, data were subjected to statistical analysis with ANOVA and Fishers PLSD tests (Statview 4.0; Abacus Concepts Inc., USA) and statistical significance accepted when p<0.05.

Results

FP receptor expression in human endometrium demonstrated a distinctive localisation pattern across the cycle. FP receptor localised to glandular epithelial cells in only mid- and late-proliferative stages of the menstrual cycle and was absent in all other biopsies examined. Only occasional expression was observed in perivascular cells and was independent of the cycle stage. In uterine adenocarcinoma biopsies FP receptor expression was also localised to epithelial cells. Epithelial cell expression of FP receptor was observed in all differentiation types with no discernible change in pattern between poor, moderately or well differentiated samples.

Quantification of FP receptor mRNA expression in both endometrial and adenocarcinoma samples was determined by Taqman quantitative RT-PCR. A significant increase in relative FP receptor expression was observed in mid- to late-proliferative endometrium (0.40±0.02; p≦0.05) when compared to early proliferative (0.06±0.02) and all secretory phases of the menstrual cycle (0.07±0.01). Within human adenocarcinoma samples relative FP receptor mRNA expression was significantly increased (116.3±63.6) compared to cycle endometrium (0.15±0.04). No correlation was observed between the different grades of adenocarcinoma, however, a large variance was observed within these tissues.

Human endometrium produced a concentration-dependent increase in total InsP production following PGF_{2α} treatment. Maximal InsP mobilisation was observed with PGF_{2α} 100 nM and inhibited by pre-treatment with U73122 2 μM, an inhibitor of PLC. Total inositol phosphate production was also measured in Ishikawa cells following treatment with PGF_{2α}. PGF_{2α} produced a concentration dependent increase in total InsP with PGF_{2α} 100 nM producing a maximum of 29 cpm/mg protein.

Treatment of Ishikawa cells with PGF_{2α} 100 nM induced phosphorylation of extracellular regulated kinase (ERK) in Ishikawa cells. Treatment for 10 min with PGF_{2α} 100 nM induced 6.6±0.7 fold increase in phosphorylated ERK intensity compared to native ERK.

The proliferative effect of PGF_{2α} in Ishikawa cells was determined by measuring incorporation of BrdU. PGF_{2α} produced a concentration-dependent increase in BrdU incorporation that was maximal at 100 nM (136.3±7.48%). Pre-treatment of cells with an ERK1/2 inhibitor (PD98059 50 μM) produced a reduction in basal proliferation (84.0±5.8%) although PGF_{2α} still produced a concentration-dependent increase in proliferation in the presence of PD98059 50 μM (100 nM PGF_{2α}-induced 119.4±11.2% control BrdU incorporation). Pre-incubation with an inhibitor of PLCβ (U73122 2 μM) also produced a slight reduction in basal proliferation (93.9±1.9%), and in addition, inhibited the concentration-dependent increase in proliferation following PGF_{2α} (106.1±6.6% BrdU incorporation by 100 nM PGF_{2α}.

Discussion

We have demonstrated increased expression of FP receptor in epithelial cells from proliferative endometrium and different grades of adenocarcinoma. Moreover, we have quantified and demonstrated functional FP receptor expression in Ishikawa cells of human endometrial adenocarcinoma origin. In Ishikawa cells, PGF_{2α} induced IP3 production and phosphorylation of p42/p44 MAPK (ERK). Additionally, following overnight incubation with PGF_{2α}, Ishiakawa cells demonstrated increased cell proliferation that was inhibited by PLC blockade.

PGF_{2α} has not previously been demonstrated to have a role in cell proliferation of mammalian tissues. Studies to identify whether PGF_{2α} was involved in colon adenocarcinomas demonstrated a lack of proliferation by PGF_{2α} in HCT-8 and HT-29 human colon adenocarcinoma cell lines. FP receptor knockout mice do not demonstrate any abnormalities in endometrial physiology but instead demonstrate failures in myometrial function with females being unable to deliver pups at term (22).

The data disclosed herein is the first demonstration of the proliferative effects of PGF_{2α} in human endometrial epithelial cells. Maximal FP receptor expression is observed in mid-late proliferative endometrium when oestrogen levels are elevated.

Without being bound by theory, it believed that the high FP receptor expression observed in adenocarcinoma samples could relate to levels of progesterone receptors where the ratio of PR_{A}:PR_{B} may be important in endometrial cancer (23). In reproductive tissue carcinomas, such as breast, endometrium and ovary, oestrogens promote cell proliferation and are implicated in disease progression (24). In the endometrium, progesterone opposes the proliferative effect of oestrogen by promoting cell differentiation and progestin treatment has proved beneficial in the management of endometrial cancer (23, 25). However, therapeutic benefit is observed only when these tumours express functional progesterone receptors (PR) (25) and combining progestins with oestrogen in hormone replacement therapy (HRT), to increase PR expression, has been found to decrease the risk of endometrial cancer (26, 27). As such, loss of responsiveness to progesterone could result in loss of progesterone inhibition and explain the observed increase in FP receptor expression observed in uterine adenocarcinoma.

Ishikawa, an endometrial epithelial cell, expresses greater levels of FP receptor than normal endometrium and is responsive to PGF_{2α}. We observed mobilisation of InsP and phosphorylation of ERK in these cells following PGF_{2α} as has been-previously identified (28, 29). Moreover, PGF_{2α} induced increased proliferation in Ishikawa cells that was sensitive to PLC blockade. This is the first documented report showing epithelial cell proliferation in response to PGF_{2α}. These observations support the role of PGF_{2α} in normal epithelial cell proliferation and endometrial adenocarcinoma where FP receptor expression is increased, and in menorrhagia.

In summary, this is the first demonstration of FP receptor expression and localisation with human endometrial tissue and human endometrial adenocarcinomas. We have demonstrated epithelial cell expression for FP receptor and have also demonstrated increased proliferation in endometrial epithelial cells, Ishikawa, following PGF_{2α}. These studies clearly indicate a role for PGF_{2α} in epithelial cell proliferation. These studies further suggest that PGF_{2α} has a role in menorrhagia, and that an agent which prevents PGF_{2α} having its effect on the FP receptor, could be used to combat menorrhagia.

### EXAMPLE 2

### Expression of FP Receptors in Uterine Tissue of Women with Menorrhagia Compared to Women with no Menorrhagia

Uterine tissue was collected by biopsy from women with a known indication of menorrhagia and women who have normal uterine function and the tissue was assessed by immunocytochemistry for the expression of the FP receptor. Briefly, tissue samples were collected, fixed, wax-embedded and sectioned, and immunohistochemical staining was performed, using standard techniques as described in Example 1 of PCT/GB02/04549 and in Example 3 below. The anti-FP receptor antibody used for the immunocytochemistry was a polyclonal antibody from Cayman Chemicals (distributed by Alexis Corporation Europe, Nottingham, UK, Catalogue number 101802), and was used at a 1/50 dilution.

As shown in panel A of FIG. 6, expression of the FP receptor is elevated in endometrial tissue from women with a known history of menorrhagia, compared to tissue from women with normal blood loss (B). The staining indicates that the FP receptor is specifically expressed in the glandular epithelial cells of menorrhagic tissue but not of normal tissue.

Hence in women with menorrhagia, it should prove beneficial to treat with agents that prevent PGF_{2α} having its effect on the FP receptor, such as FP receptor antagonists, in order to block the signalling pathway and ultimately transcription of target genes that may mediate vascular function/dysfunction and excessive bleeding.

### EXAMPLE 3

### Elevated Expression of EP2 and EP4 Receptors in Endometrium of Menorrhagic Women Compared to Control Women

Methods

Endometrial sections (5 μm) collected from two women classed as control (with <80 ml blood loss per cycle) or menorrhagic (with >80 ml blood loss per cycle) were dewaxed in xylene and rehydrated using decreasing grades of ethanol. After rinsing in PBS, endogenous peroxidase activity was quenched with 3% H₂O₂ in methanol. Non-immune swine serum (10% serum in PBS) was applied for 20 min before overnight incubation at 4° C. with primary antibody. An avidin-biotin peroxidase detection system was then applied (DAKO Ltd, UK) with 3,3′-diaminobenzidine (DAB) as the chromagen. Sections were counter stained with Harris's haematoxylin before mounting. The primary antibodies used in this study were raised in rabbits against human EP2 or EP4 receptor peptide sequences (Cayman Chemicals, USA). The antibody was used at a 1:250 dilution. All treatments were carried out at room temperature unless otherwise specified.

Results

Staining for both EP2 and EP4 receptors was localised in the glandular epithelial cells and endothelial cells. Lower intensity of staining was observed in the endometrial samples collected from the woman with normal bleeding pattern as compared with endometrium collected with women suffering from menorrhagia. This indicates a higher expression pattern of the two receptors in the latter group of women.

The results are shown in FIG. 7.

### EXAMPLE 4

### Treatment of Menorrhagia with an FP Receptor Antagonist

A woman presents to her physician with symptoms of menorrhagia. The physician diagnoses menorrhagia. She is administered AL-3138 or AL-8810 at a dosing quantity and frequency such that the therapeutic level of active agent at the site of treatment is maintained at a level ideally EC₉₀ but preferably not less than EC₅₀ throughout the treatment period. The treatment is delivered orally or more locally depending on patient acceptability, avoidance of side effects and systemic bioavailability.

### EXAMPLE 5

### Treatment of Menorrhagia with an FP Receptor Antagonist and an EP2 Receptor Antagonist

A woman presents to her physician with symptoms of menorrhagia. The physician diagnoses menorrhagia. She is administered AL-3138 or AL-8810 and AH6809 at a dosing quantity and frequency such that the therapeutic level of active agents at the site of treatment is maintained at a level ideally EC₉₀ but preferably not less than EC₅₀ throughout the treatment period. The treatment is delivered orally or more locally depending on patient acceptability, avoidance of side effects and systemic bioavailability.

### EXAMPLE 6

### Treatment of Menorrhagia with an FP Receptor Antagonist and an EP4 Receptor Antagonist

A woman presents to her physician with symptoms of menorrhagia. The physician diagnoses menorrhagia. She is administered AL-3138 or AL-8810 and AH23848B at a dosing quantity and frequency such that the therapeutic level of active agents at the site of treatment is maintained at a level ideally EC₉₀ but preferably not less than EC₅₀ throughout the treatment period. The treatment is delivered orally or more locally depending on patient acceptability, avoidance of side effects and systemic bioavailability.

### EXAMPLE 7

### Suppository

| | mg/suppository |
|---|---|
| AL-3138 or AL-8821 (63 μm)* | 250 |
| Hard Fat, BP (Witepsol H15 - Dynamit Nobel) | 1770 |
| | 2020 |

*The antagonist AL-3138 or AL-8821 is typically used as a powder wherein at least 90% of the particles are of 63 μm diameter or less.

One fifth of the Witepsol H15 is melted in a steam-jacketed pan at 45° C. maximum. The active ingredient is sifted through a 200 μm sieve and added to the molten base with mixing, using a silverson fitted with a cutting head, until a smooth dispersion is achieved. Maintaining the mixture at 45° C., the remaining Witepsol H15 is added to the suspension and stirred to ensure a homogenous mix. The entire suspension is passed through a 250 μm stainless steel screen and, with continuous stirring, is allowed to cool to 40° C. At a temperature of 38° C. to 40° C. 2.02 g of the mixture is filled into suitable plastic moulds. The suppositories are allowed to cool to room temperature.

### EXAMPLE 8

### Pessaries

| | mg/pessary |
|---|---|
| AL-3138 or AL-8821 | 250 |
| Anhydrate Dextrose | 380 |
| Potato Starch | 363 |
| Magnesium Stearate | 7 |
| | 1000 |

The above ingredients are mixed directly and pessaries prepared by direct compression of the resulting mixture.

### EXAMPLE 9

### Vaginal Ring

A vaginal ring containing AL-3138 or AL-8821 is produced using core extrusion technology.

### EXAMPLE 10

### Vaginal Ring

A vaginal ring containing AL-3138 or AL-8821 and AH 6809 is produced using core extrusion technology.

### EXAMPLE 11

### Intrauterine Device

An intrauterine device containing AL-3138 or AL-8821 is produced using standard technology.

### EXAMPLE 12

### Intrauterine Device

An intrauterine device containing AL-3138 or AL-8821 and AH23848B is produced using standard technology.

### EXAMPLE 13

### Tampon

A tampon for treating menorrhagia is produced by impregnating a standard tampon with an effective dose of AL-3138 or AL-8821.

### EXAMPLE 14

### Tampon

A tampon is produced by impregnating a standard tampon with an effective dose of AL-3138 or AL-8821 and AH6809 or AH23848B.

### REFERENCES FOR EXAMPLE 1

1. Narumiya, S. and FitzGerald, G. A. Genetic and pharmacological analysis of prostanoid receptor function. J Clin Invest, 108: 25-30, 2001.

2. Coleman, R. A., Smith, W. L., and Narumiya, S. International Union of Pharmacology classification of prostanoid receptors: properties, distribution, and structure of the receptors and their subtypes. Pharmacol Rev, 46: 205-229, 1994.

3. Vane, J. R., Bakhle, Y. S., and Botting, R. M. Cyclooxygenases 1 and 2. Annu Rev Pharmacol Toxicol, 38: 97-120, 1998.

4. Yokoyama, C., Miyata, A., Ihara, H., Ullrich, V., and Tanabe, T. Molecular cloning of human platelet thromboxane A synthase. Biochem Biophys Res Commun, 178: 1479-1484, 1991.

5. Suzuki-Yamamoto, T., Nishizawa, M., Fukui, M., Okuda-Ashitaka, E., Nakajima, T., Ito, S., and Watanabe, K. cDNA cloning, expression and characterization of human prostaglandin F synthase. FEBS Lett, 462: 335-340, 1999.

6. Miyata, A., Hara, S., Yokoyama, C., Inoue, H., Ullrich, V., and Tanabe, T. Molecular cloning and expression of human prostacyclin synthase. Biochem Biophys Res Commun, 200: 1728-1734, 1994.

7. Kanaoka, Y., Ago, H., Inagaki, E., Nanayama, T., Miyano, M., Kikuno, R., Fujii, Y., Eguchi, N., Toh, H., Urade, Y., and Hayaishi, O. Cloning and crystal structure of hematopoietic prostaglandin D synthase. Cell, 90: 1085-1095, 1997.

8. Forsberg, L., Leeb, L., Thoren, S., Morgenstern, R., and Jakobsson, P. Human glutathione dependent prostaglandin E synthase: gene structure and regulation. FEBS Lett, 471: 78-82, 2000.

9. Tsujii, M., Kawano, S., Tsuji, S., Sawaoka, H., Hori, M., and DuBois, R. N. Cyclooxygenase regulates angiogenesis induced by colon cancer cells. Cell, 93: 705-716, 1998.

10. Van Voorhis, B. J., Huettner, P. C., Clark, M. R., and Hill, J. A. Immunohistochemical localization of prostaglandin H synthase in the female reproductive tract and endometriosis. Am J Obstet Gynecol, 163: 57-62, 1990.

11. Rees, M. C., Parry, D. M., Anderson, A. B., and Turnbull, A. C. Immunohistochemical localisation of cyclooxygenase in the human uterus. Prostaglandins, 23: 207-214, 1982.

12. Rees, M. C., Anderson, A. B., Demers, L. M., and Turnbull, A. C. Endometrial and myometrial prostaglandin release during the menstrual cycle in relation to menstrual blood loss. J Clin Endocrinol Metab, 58: 813-818, 1984.

13. Jones, R. L., Kelly, R. W., and Critchley, H. O. D. Chemokine and cyclooxygenase-2 expression in human endometrium coincides with leukocyte accumulation. Hum Reprod, 12: 1300-1306, 1997.

14. Marions, L. and Danielsson, K. G. Expression of cyclo-oxygenase in human endometrium during the implantation period. Mol Hum Reprod, 5: 961-965, 1999.

15. Abel, M. H. and Baird, D. T. The effect of 17b-estradiol and progesterone on prostaglandin production by human endometrium maintained in organ culture. Endocrinology, 106: 1599-1606, 1980.

16. Fortier, M. A., Guilbault, L. A., and Grasso, F. Specific properties of epithelial and stromal cells from the endometrium of cows. J Reprod Fertil, 83: 239-248., 1988.

17. Ou, C. W., Chen, Z. Q., Qi, S., and Lye, S. J. Expression and regulation of the messenger ribonucleic acid encoding the prostaglandin F(2alpha) receptor in the rat myometrium during pregnancy and labor. Am J Obstet Gynecol, 182: 919-925, 2000.

18. Dong, Y. L. and Yallampalli, C. Pregnancy and exogenous steroid treatments modulate the expression of relaxant EP(2) and contractile FP 25 receptors in the rat uterus. Biol Reprod, 62: 533-539, 2000.

19. Hoyer, P. B., Marion, S. L., Stine, I., Rueda, B. R., Hamernik, D. L., Regan, J. W., and Wise, M. E. Ovine prostaglandin F2alpha receptor: steroid influence on steady-state levels of luteal mRNA. Endocrine, 10: 105-111, 1999.

20. Noyes, R. W., Hertig, A. T., and Rock, J. Dating the endometrial biopsy. Fertil Steril, 1: 3-25, 1950.

21. Berg, K. A., Clarke, W. P., Sailstad, C., Saltzman, A., and Maayani, S. Signal transduction differences between 5-hydroxytryptamine type 2A and type 2C receptor systems. Mol Pharmacol, 46: 477-484, 1994.

22. Sugimoto, Y., Yamasaki, A., Segi, E., Tsuboi, K., Aze, Y., Nishimur, T., Oida, H., Yoshida, N., Tanaka, T., Katsuyama, M., Hasumoto, K., Murata, T., Hirata, M., Ushikubi, F., Negishi, M., Ichikawa, A., and Narumiya, S. Failure of parturition in mice lacking the prostaglandin F receptor. Science, 277: 681-683, 1997.

23. Kumar, N. S., Richer, J., Owen, G., Litman, E., Horwitz, K. B., and Leslie, K. K. Selective down-regulation of progesterone receptor isoform B in poorly differentiated human endometrial cancer cells: implications for unopposed estrogen action. Cancer Res, 58: 1860-1865, 1998.

24. Persson, I. Estrogens in the causation of breast, endometrial and ovarian cancers—evidence and hypotheses from epidemiological findings. J Steroid Biochem Mol Biol, 74: 357-364, 2000.

25. Sasaki, M., Dharia, A., Oh, B. R., Tanaka, Y., Fujimoto, S., and Dahiya, R. Progesterone receptor B gene inactivation and CpG hypermethylation in human uterine endometrial cancer. Cancer Res, 61: 97-102, 2001.

26. Dai, D., Kumar, N. S., Wolf, D. M., and Leslie, K. K. Molecular tools to reestablish progestin control of endometrial cancer cell proliferation. Am J Obstet Gynecol, 184: 790-797, 2001.

27. Lim, C. S., Baumann, C. T., Htun, H., Xian, W., Irie, M., Smith, C. L., and Hager, G. L. Differential localization and activity of the A- and B-forms of the human progesterone receptor using green fluorescent protein chimeras. Mol Endocrinol, 13: 366-375, 1999.

28. Chen, D. B., Westfall, S. D., Fong, H. W., Roberson, M. S., and Davis, J. S. Prostaglandin F2alpha stimulates the Raf/MEK1/mitogen-activated protein kinase signaling cascade in bovine luteal cells. Endocrinology, 139: 3876-3885, 1998.

29. Stocco, C. O., Lau, L. F., and Gibori, G. A calcium/calmodulin-dependent activation of ERK1/2 mediates JunD phosphorylation and induction of nur77 and 20alpha-hsd genes by prostaglandin F2alpha in ovarian cells. J Biol Chem, 277: 3293-3302, 2002.

### FURTHER REFERENCES

Ashby, B. (1998) Co-expression of prostaglandin receptors with opposite effects: a model for homeostatic control of autocrine and paracrine signalling. Biochem Pharmacol 55: 239-246.
Coleman, R A, Smith, W L, Narumiya, S. (1994) International Union of Pharmacology classification of prostanoid receptors: properties, distribution, and structure of the receptors and their subtypes. Pharmacol Rev 46: 205-229.
DeWitt, D L. (1991) Prostaglandin endoperoxide synthase: regulation of enzyme expression. Biochim Biophys Acta 1083: 121-134.
Herschman, H R. (1996) Prostaglandin synthase 2. Biochim Biophys Acta 1299: 125-140.
Subbaramaiah, K, Telang, N, Ramonetti, J T, Araki, R, DeVito, B, Weksler, B B, Dannenberg, A J. (1996) Transcription of cyclooxygenase-2 is enhanced in transformed mammary epithelial cells. Cancer Res 56: 4424-4429.

1. A method of treating or preventing menorrhagia in a female individual, the method comprising administering to the individual at least one agent that prevents PGF_{2α} having its effect on the FP receptor.

2. A method according to claim 1 wherein the agent that prevents PGF_{2α} having its effect on the FP receptor prevents or reduces the binding of PGF_{2α} to the FP receptor.

3. A method according to claim 1 wherein the agent that prevents PGF_{2α} having its effect on the FP receptor affects the interaction between PGF_{2α} and the FP receptor, or the interaction between the FP receptor and the associated G_{αq} protein, thus inhibiting or disrupting a PGF_{2α}-FP mediated signal transduction pathway.

4. A method according to any of claim 1 wherein the agent is an antagonist of the FP receptor.

5. A method according to claim 4 wherein the FP receptor antagonist is any one or more of PGF_{2α} dimethyl amide; PGF_{2α} dimethyl amine; AL-8810 ((5Z,13E)-(9S,11S,15R)-9,15-dihydroxy-11-fluoro-15-(2-indanyl)-16,17,18,19,20-pentanor-5,13-prostadienoic acid); AL-3138 (11-deoxy-16-fluoro PGF_{2α}); phloretin; glibenclamide; ridogrel; PHG113; PCP-1 (rvkfksqqhrqgrshhlem); PCP-2 (rkavlknlyklasqccgvhvislhiwelssiknslkvaaisespvaeksast); PCP-3 (clseeakearrindeierqlrrdkrdarre-NH₂); PCP-4 (kdtilqlnlkeynlv-NH₂); PCP-8 (ilghrdyk); PCP-10 (wedrfyll); PCP-13 (ILGHRDYK); PCP-14 (YQDRFYLL); (ILAHRDYK); PCP-13.7 (ILAHRDYK); PCP-13.8 (ILaHRDYK); PCP-13.11 (ILGFRDYK); PCP-13.13 (ILGHKDYK); PCP-13.14 (ILGHRNYK); PCP-13.18 (ILGHQDYK); PCP-13.20 (ILGHRDY-amide); PCP-13.21 (ILGHRDYK-amide); PCP-13.22 (ILGWRDYK); PCP-13.24 (ILGXRDYK); and PCP-15 (SNVLCSIF).

6. A method according to claim 1 wherein the agent is an antagonist of PGF_{2α}.

7. A method according to claim 6 wherein the PGF_{2α} antagonist is an anti-PGF_{2α} antibody.

8. A method according to claim 1 further comprising administering to the individual one or more of an inhibitor of PGES and/or an antagonist of EP2 or EP4.

9. A method according to claim 8 wherein the antagonist of EP2 or EP4 is AH6809, an omega-substituted prostaglandin E derivative, AH23848B, AH22921X, IFTSYLECL, IFASYECL, IFTSAECL, IFTSYEAL, ILASYECL, IFTSTDCL, TSYEAL (with 4-biphenylalanine), TSYEAL (with homophenylalanine), a 5-thia-prostaglandin E derivative, 5-butyl-2,4-dihydro-4-[[2′-[N-(3-chloro-2-thiophenecarbonyl)sulfamoyl]biphenyl-4-yl]methyl]-2-{2-(trifluoromethyl)phenyl]-1,2,4-triazol-3-one potassium salt, 5-butyl-2,4-dihydro-4-[[2′-[N-(2-methyl-3-furoyl)sulfamoyl]biphenyl-4-yl]methyl]-2-{2-(trifluoromethyl)phenyl]-1,2,4-triazol-3-one, 5-butyl-2,4-dihydro-4-[[2′-[N-(3-methyl-2-thiophenecarbonyl)sulfamoyl]biphenyl-4-yl]methyl]-2-{2-(trifluoromethyl)phenyl]-1,2,4-triazol-3-one, 5-butyl-2,4-dihydro-4-[[2′-[N-(2-thiophenecarbonyl)sulfamoyl]biphenyl-4-yl]methyl]-2-{2-(trifluoromethyl)phenyl]-1,2,4-triazol-3-one, or 5-butyl-2,4-dihydro-4-[[2′-[N-[2-(methypyrrole)carbonyl]sulfamoyl]biphenyl-4-yl]methyl]-2-{2-(trifluoromethyl)phenyl]-1,2,4-triazol-3-one.

10. Use of at least one agent that prevents PGF_{2α} having its effect on the FP receptor, in the manufacture of a medicament for treating or preventing menorrhagia in a female individual.

11. Use according to claim 10, wherein the individual is administered one or more of an inhibitor of PGES and/or an antagonist of EP2 or EP4.

12. Use of a combination of at least one agent that prevents PGF_{2α}, having its effect on the FP receptor, and one or more of an inhibitor of PGES and/or an antagonist of EP2 or EP4, in the manufacture of a medicament for treating or preventing menorrhagia in a female individual.

13. Use of one or more of an inhibitor of PGES and/or an antagonist of EP2 or EP4 in the manufacture of a medicament for treating or preventing menorrhagia in a female individual, wherein the female individual is administered at least one agent that prevents PGF_{2α} having its effect on the FP receptor.

14. A pharmaceutical composition comprising at least one agent that prevents PGF_{2α} having its effect on the FP receptor for treating or preventing menorrhagia in a female individual.

15. A pharmaceutical composition according to claim 14 further comprising one or-more of an inhibitor of PGES and/or an antagonist of EP2 or EP4.

16. A vaginal ring or a tampon or an intrauterine device comprising at least one agent that prevents PGF_{2α} having its effect on the FP receptor.

17. A vaginal ring or a tampon or an intrauterine device according to claim 16 further comprising one or more of an inhibitor of PGES and/or an antagonist of EP2 or EP4.

18. A use according to claim 10, wherein the agent that prevents PGF_{2α} having its effect on the FP receptor (i) prevents or reduces the binding of PGF_{2α} to the FP receptor, (ii) affects the interaction between PGF_{2α} and the FP receptor, or the interaction between the FP receptor and the associated G_{αq} protein, thus inhibiting or disrupting a PGF_{2α}-FP mediated signal transduction pathway, (iii) is an antagonist of the FP receptor, (iv) is an antagonist of PGF_{2α}, or (v) is an anti-PGF_{2α} antibody.

19. Use according to claim 11, wherein the antagonist of EP2 or EP4 is selected from the group of AH6809, an omega-substituted prostaglandin E derivative, AH23848B, AH22921X, IFTSYLECL, IFASYECL, IFTSAECL, IFTSYEAL, ILASYECL, IFTSTDCL, TSYEAL (with 4-biphenylalanine), TSYEAL (with homophenylalanine), a 5-thia-prostaglandin E derivative, 5-butyl-2,4-dihydro-4-[[2′-[N-(3-chloro-2-thiophenecarbonyl)sulfamoyl]biphenyl-4-yl]methyl]-2-{2-(trifluoromethyl)phenyl]-1,2,4-triazol-3-one potassium salt, 5-butyl-2,4-dihydro-4-[[2′-[N-(2-methyl-3-furoyl)sulfamoyl]biphenyl-4-yl]methyl]-2-{2-(trifluoromethyl)phenyl]-1,2,4-triazol-3-one, 5-butyl-2,4-dihydro-4-[[2′-[N-(3-methyl-2-thiophenecarbonyl)sulfamoyl]biphenyl-4-yl]methyl]-2-{2-(trifluoromethyl)phenyl]-1,2,4-triazol-3-one, 5-butyl-2,4-dihydro-4-[[2′-[N-(2-thiophenecarbonyl)sulfamoyl]biphenyl-4-yl]methyl]-2-{2-(trifluoromethyl)phenyl]-1,2,4-triazol-3-one, or 5-butyl-2,4-dihydro-4-[[2′-[N-[2-(methypyrrole)carbonyl]sulfamoyl]biphenyl-4-yl]methyl]-2-{2-(trifluoromethyl)phenyl]-1,2,4-triazol-3-one.

20. A composition comprising at least one agent that prevents PGF_{2α} having its effect on the FP receptor, and one or more of an inhibitor of PGES and/or an antagonist of EP2 or EP4.

21. A pharmaceutical composition comprising at least one agent that prevents PGF_{2α} having its effect on the FP receptor, and one or more of an inhibitor of PGES and/or an antagonist of EP2 or EP4, and a pharmaceutically acceptable carrier.

22. A composition according to claim 20 for use in medicine.

23. Use according to claim 10 wherein the agent that prevents PGF_{2α} having its effect on the FP receptor is an antagonist of the FP receptor is selected from the group of any one or more of PGF_{2α} dimethyl amide; PGF_{2α} dimethyl amine; AL-8810 ((5Z,13E)-(9S,11S,15R)-9,15-dihydroxy-11-fluoro-15-(2-indanyl)-16,17,18,19,20-pentanor-5,13-prostadienoic acid); AL-3138 (11-deoxy-16-fluoro PGF_{2α}); phloretin; glibenclamide; ridogrel; PHG113; PCP-1 (rvkfksqqhrqgrshhlem); PCP-2 (rkavlknlyklasqccgvhvislhiwelssiknslkvaaisespvaeksast); PCP-3 (clseeakearrindeierqlrrdkrdarre-NH₂); PCP-4 (kdtilqlnlkeynlv-NH₂); PCP-8 (ilghrdyk); PCP-10 (wedrfyll); PCP-13 (ILGHRDYK); PCP-14 (YQDRFYLL); (ILAHRDYK); PCP-13.7 (ILAHRDYK); PCP-13.8 (ILaHRDYK) ; PCP-13.11 (ILGFRDYK); PCP-13.13 (ILGHKDYK); PCP-13.14 (ILGHRNYK); PCP-13.18 (ILGHQDYK); PCP-13.20 (ILGHRDY-amide); PCP-13.21 (ILGHRDYK-amide); PCP-13.22 (ILGWRDYK); PCP-13.24 (ILGXRDYK); and PCP-15 (SNVLCSIF).

24. A pharmaceutical composition according to claim 14 wherein the agent that prevents PGF_{2α} having its effect on the FP receptor (i) prevents or reduces the binding of PGF_{2α} to the FP receptor, (ii) affects the interaction between PGF_{2α} and the FP receptor, or the interaction between the FP receptor and the associated G_{αq} protein, thus inhibiting or disrupting a PGF_{2α}-FP mediated signal transduction pathway, (iii) is an antagonist of the FP receptor, (iv) is an antagonist of PGF_{2α}, or (v) is an anti-PGF_{2α} antibody.

25. A pharmaceutical composition according to claim 14 wherein the agent that prevents PGF_{2α} having its effect on the FP receptor is an antagonist of the FP receptor is selected from the group of any one or more of PGF_{2α} dimethyl amide; PGF_{2α} dimethyl amine; AL-8810 ((5Z,13E)-(9S,11S,15R)-9,15-dihydroxy-11-fluoro-15-(2-indanyl)-16,17,18,19,20-pentanor-5,13-prostadienoic acid); AL-3138 (11-deoxy-16-fluoro PGF_{2α}); phloretin; glibenclamide; ridogrel; PHG113; PCP-1 (rvkfksqqhrqgrshhlem); PCP-2 (rkavlknlyklasqccgvhvislhiwelssiknslkvaaisespvaeksast); PCP-3 (clseeakearrindeierqlrrdkrdarre-NH₂); PCP-4 (kdtilqlnlkeynlv-NH₂); PCP-8 (ilghrdyk); PCP-10 (wedrfyll); PCP-13 (ILGHRDYK); PCP-14 (YQDRFYLL); (ILAHRDYK); PCP-13.7 (ILAHRDYK); PCP-13.8 (ILaHRDYK) ; PCP-13.11 (ILGFRDYK); PCP-13.13 (ILGHKDYK); PCP-13.14 (ILGHRNYK); PCP-13.18 (ILGHQDYK); PCP-13.20 (ILGHRDY-amide); PCP-13.21 (ILGHRDYK-amide); PCP-13.22 (ILGWRDYK); PCP-13.24 (ILGXRDYK); and PCP-15 (SNVLCSIF).

26. A vaginal ring or a tampon or an intrauterine device according to claim 16 wherein the at least one agent that prevents PGF_{2α} having its effect on the FP receptor (i) prevents or reduces the binding of PGF_{2α} to the FP receptor, (ii) affects the interaction between PGF_{2α} and the FP receptor, or the interaction between the FP receptor and the associated G_{α}q protein, thus inhibiting or disrupting a PGF_{2α}-FP mediated signal transduction pathway, (iii) is an antagonist of the FP receptor, (iv) is an antagonist of PGF_{2α}, or (v) is an anti-PGF_{2α} antibody.

27. A vaginal ring or a tampon or an intrauterine device according to claim 16 wherein the agent that prevents PGF_{2α} having its effect on the FP receptor is an antagonist of the FP receptor is selected from the group of any one or more of PGF_{2α} dimethyl amide; PGF_{2α} dimethyl amine; AL-8810 ((5Z,13E)-(9S, 11S,15R)-9,15-dihydroxy-11-fluoro-15-(2-indanyl)-16,17,18,19,20-pentanor-5,13-prostadienoic acid); AL-3138 (11-deoxy-16-fluoro PGF_{2α}); phloretin; glibenclamide; ridogrel; PHG113; PCP-1 (rvkfksqqhrqgrshhlem); PCP-2 (rkavlknlyklasqccgvhvislhiwelssiknslkvaaisespvaeksast); PCP-3 ( clseeakearrindeierqlrrdkrdarre-NH₂); PCP-4 (kdtilqlnlkeynlv-NH₂); PCP-8 (ilghrdyk); PCP-10 (wedrfyll); PCP-13 (ILGHRDYK); PCP-14 (YQDRFYLL); (ILAHRDYK); PCP-13.7 (ILAHRDYK); PCP-13.8 (ILaHRDYK); PCP-13.11 (ILGFRDYK); PCP-13.13 (ILGHKDYK); PCP-13.14 (ILGHRNYK); PCP-13.18 (ILGHQDYK); PCP-13.20 (ILGHRDY-amide); PCP-13.21 (ILGHRDYK-amide); PCP-13.22 (ILGWRDYK); PCP-13.24 (ILGXRDYK); and PCP-15 (SNVLCSIF).

28. A pharmaceutical composition according to claim 15 wherein the antagonist of EP2 or EP4 is selected from the group of AH6809, an omega-substituted prostaglandin E derivative, AH23848B, AH22921X, IFTSYLECL, IFASYECL, IFTSAECL, IFTSYEAL, ILASYECL, IFTSTDCL, TSYEAL (with 4-biphenylalanine), TSYEAL (with homophenylalanine), a 5-thia-prostaglandin E derivative, 5-butyl-2,4-dihydro-4-[[2′-[N-(3-chloro-2-thiophenecarbonyl)sulfamoyl]biphenyl-4-yl]methyl]-2-{2-(trifluoromethyl)phenyl]-1,2,4-triazol-3-one potassium salt, 5-butyl-2,4-dihydro-4-[[2′-[N-(2-methyl-3-furoyl)sulfamoyl]biphenyl-4-yl]methyl]-2-{2-(trifluoromethyl)phenyl]-1,2,4-triazol-3-one, 5-butyl-2,4-dihydro-4-[[2′-[N-(3-methyl-2-thiophenecarbonyl)sulfamoyl]biphenyl-4-yl]methyl]-2-{2-(trifluoromethyl)phenyl]-1,2,4-triazol-3-one, 5-butyl-2,4-dihydro-4-[[2′-[N-(2-thiophenecarbonyl)sulfamoyl]biphenyl-4-yl]methyl]-2-{2-(trifluoromethyl)phenyl]-1,2,4-triazol-3-one, or 5-butyl-2,4-dihydro-4-[[2′-[N-[2-(methypyrrole)carbonyl]sulfamoyl]biphenyl-4-yl]methyl]-2-{2-(trifluoromethyl)phenyl]-1,2,4-triazol-3-one.

29. A vaginal ring or a tampon or an intrauterine device according to claim 17 wherein the antagonist of EP2 or EP4 is selected from the group of AH6809, an omega-substituted prostaglandin E derivative, AH23848B, AH22921X, IFTSYLECL, IFASYECL, IFTSAECL, IFTSYEAL, ILASYECL, IFTSTDCL, TSYEAL (with 4-biphenylalanine), TSYEAL (with homophenylalanine), a 5-thia-prostaglandin E derivative, 5-butyl-2,4-dihydro-4-[[2′-[N-(3-chloro-2-thiophenecarbonyl)sulfamoyl]biphenyl-4-yl]methyl]-2-{2-(trifluoromethyl)phenyl]-1,2,4-triazol-3-one potassium salt, 5-butyl-2,4-dihydro-4-[[2′-[N-(2-methyl-3-furoyl)sulfamoyl]biphenyl-4-yl]methyl]-2-{2-(trifluoromethyl)phenyl]-1,2,4-triazol-3-one, 5-butyl-2,4-dihydro-4-[[2′-[N-(3-methyl-2-thiophenecarbonyl)sulfamoyl]biphenyl-4-yl]methyl]-2-{2-(trifluoromethyl)phenyl]-1,2,4-triazol-3-one, 5-butyl-2,4-dihydro-4-[[2′-[N-(2-thiophenecarbonyl)sulfamoyl]biphenyl-4-yl]methyl]-2-{2-(trifluoromethyl)phenyl]-1,2,4-triazol-3-one, or 5-butyl-2,4-dihydro-4-[[2′-[N-[2-(methypyrrole)carbonyl]sulfamoyl]biphenyl-4-yl]methyl]-2-{2-(trifluoromethyl)phenyl]-1,2,4-triazol-3-one.
